(19) United States
(12) Patent Application Publication
(10) Pub. No.: US 2026/0217531 A1
(43) Pub. Date: Jul. 30, 2026

(54) METHOD OF MANUFACTURE FOR HYDRAZINE COMPOSITION

(71) Applicant: AEROJET ROCKETDYNE, INC., Melbourne, FL (US)

(72) Inventor: Robert K. Masse, Redmond, WA (US)

(21) Appl. No.: 19/144,970

(22) PCT Filed: Jun. 21, 2023
(86) PCT No.: PCT/US2023/025837

§ 371 Date: Jul. 1, 2025

Related U.S. Application Data
(60) Provisional application No. 63/457,476, filed on Apr. 6, 2023.

(51) Int. Cl. C01B 21/16; C07C 213/08; C07C 213/10

(52) U.S. Cl. CPC C01B 21/16; C07C 213/08; C07C 213/10

(57) ABSTRACT

A method includes reacting a mixture of hydrazine hydrate and hydroxyethylhydrazine with a nitrate to produce a product mixture that includes hydrazine hydrate, hydrazinium nitrate, hydroxyethylhydrazine, and hydroxyethylhydrazinium nitrate, and distilling the product mixture to produce a final mixture that includes hydrazine, hydroxyethylhydrazinium nitrate, and hydrazinium nitrate.

### BACKGROUND

Mixtures of hydrazine, hydrazine nitrate, and hydroxyethylhydrazine nitrate provide a reduced-vapor-toxicity alternative to pure hydrazine in such end-uses as a rocket propellants. Manufacturing methods for producing such mixtures have exclusively focused on mixing high-purity hydrazine, hydroxyethyl hydrazine, and ammonium nitrate, followed by removal of ammonia via vacuum distillation and sparging with dry gas. The high cost of high-purity hydrazine and hydrazinium nitrate, however, results in a commensurate high cost of the mixture.

### SUMMARY

A method according to an example of the present disclosure includes reacting a mixture of hydrazine hydrate and hydroxyethylhydrazine with a nitrate to produce a product mixture that includes hydrazine hydrate, hydrazinium nitrate, hydroxyethylhydrazine, and hydroxyethylhydrazinium nitrate and distilling the product mixture to produce a final mixture that includes hydrazine, hydroxyethylhydrazinium nitrate, and hydrazinium nitrate.

In a further embodiment of any of the foregoing embodiments, the nitrate is selected from the group consisting of ammonium nitrate and nitric acid.

In a further embodiment of any of the foregoing embodiments, the nitrate includes ammonium nitrate.

In a further embodiment of any of the foregoing embodiments, the nitrate includes nitric acid.

In a further embodiment of any of the foregoing embodiments, the nitrate is ammonium nitrate.

In a further embodiment of any of the foregoing embodiments, the nitrate is nitric acid.

In a further embodiment of any of the foregoing embodiments, the mixture of hydrazine hydrate and hydroxyethylhydrazine is produced by first reacting ethylene oxide and an excess of hydrazine hydrate to yield an intermediate mixture that includes the hydrazine hydrate, the hydroxyethylhydrazine, and bis(hydroxyethyl) hydrazine.

A further embodiment of any of the foregoing embodiments includes distilling the intermediate mixture to substantially remove the bis(hydroxyethyl) hydrazine from the intermediate mixture to produce the mixture of hydrazine hydrate and hydroxyethylhydrazine.

In a further embodiment of any of the foregoing embodiments, the final mixture has a molar composition of 50-75% hydrazine, 15-35% hydroxyethylhydrazinium nitrate, and 5-15% hydrazinium nitrate.

A method according to an example of the present disclosure includes producing a mixture of hydrazine hydrate and hydroxyethylhydrazine by reacting ethylene oxide and an excess of hydrazine hydrate to yield an intermediate mixture that includes the hydrazine hydrate, the hydroxyethylhydrazine, and bis(hydroxyethyl) hydrazine, distilling the intermediate mixture to substantially remove the bis(hydroxyethyl) hydrazine from the intermediate mixture to produce the mixture of hydrazine hydrate and hydroxyethylhydrazine, reacting the mixture of hydrazine hydrate and hydroxyethylhydrazine with a nitrate to produce a product mixture includes that hydrazine hydrate, hydrazinium nitrate, hydroxyethylhydrazine, and hydroxyethylhydrazinium nitrate, and distilling the product mixture to yield a final mixture that includes hydrazine, hydroxyethylhydrazinium nitrate, and hydrazinium nitrate.

In a further embodiment of any of the foregoing embodiments, the nitrate is selected from the group consisting of ammonium nitrate and nitric acid.

In a further embodiment of any of the foregoing embodiments, the nitrate includes ammonium nitrate.

In a further embodiment of any of the foregoing embodiments, the nitrate includes nitric acid.

In a further embodiment of any of the foregoing embodiments, the nitrate is ammonium nitrate.

In a further embodiment of any of the foregoing embodiments, the nitrate is nitric acid.

In a further embodiment of any of the foregoing embodiments, a molar ratio of the excess of hydrazine hydrate to the ethylene oxide is from 2:1 to 9:1.

The present disclosure may include any one or more of the individual features disclosed above and/or below alone or in any combination thereof.

### BRIEF DESCRIPTION OF THE DRAWINGS

The various features and advantages of the present disclosure will become apparent to those skilled in the art from the following detailed description. The drawings that accompany the detailed description can be briefly described as follows.

FIG. 1 depicts a method of manufacturing a mixture of high-purity hydrazine, hydroxyethylhydrazine, and hydrazinium nitrate.

### DETAILED DESCRIPTION

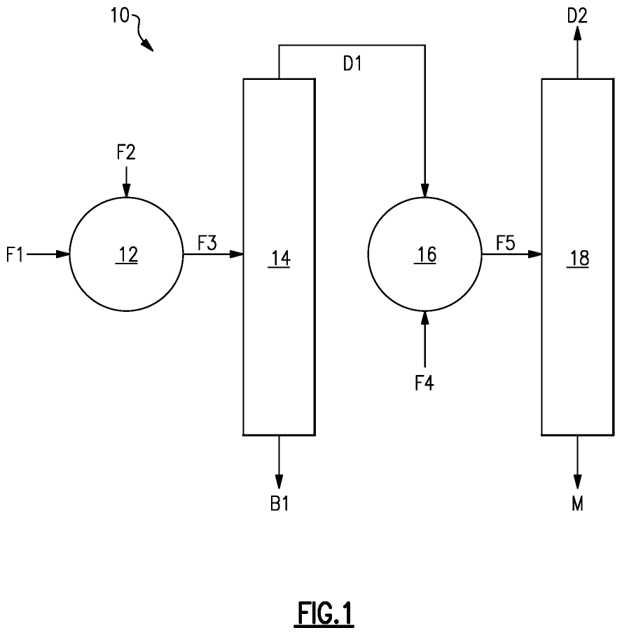

FIG. 1 schematically depicts a method 10 of manufacturing a reduced-cost mixture of high-purity hydrazine, hydroxyethylhydrazine, and hydrazinium nitrate. For example, the mixture is made from relatively low-cost feedstocks of hydrazine hydrate and ethylene oxide. As shown in FIG. 1, a feedstock F1 of hydrazine hydrate (N₂H₄·H₂O) and a feedstock F2 of ethylene oxide (EtOH) are introduced into a reactor 12. The feedstocks F1 and F2 are provided in a ratio that has a molar excess of the hydrazine hydrate in a preferred ratio between 2 and 9 parts hydrazine hydrate by mole per part EtOH (2:1 to 9:1) such that the resulting output intermediate mixture F3 includes hydrazine hydrate and reaction products hydroxyethylhydrazine (HEH), water (H₂O), and bis(hydroxyethyl) hydrazine (Bis(HE)H) according to Equation I below. The reaction conditions in the reactor 12, which may be optimized to maximize the yield of hydroxyethylhydrazine and minimize the amount of bis(hydroxyethyl) hydrazine byproduct produced, will preferably range between 3° and 100° C. in temperature, and 0.1 to 0.5 psi gauge pressure.

$${N}_{2} {H}_{4} \cdot {H}_{2} O + EtOH \to {N}_{2} {H}_{4} \cdot {H}_{2} O + HEH + {H}_{2} O + Bis ( HE ) H \qquad Equation I$$

The resulting output intermediate mixture F3 is then processed through one or more distillation columns 14. The distillate D1 that exits at the top of the distillation column 14 includes a mixture of hydrazine hydrate, hydroxyethylhydrazine, and water that is substantially free (preferably <1%) of the byproduct bis(hydroxyethyl) hydrazine. A byproduct B1 exits at the bottom of the distillation column 14. The byproduct B1 is a bis(hydroxyethyl) hydrazine-rich mixture. Optionally, the bis(hydroxyethyl) hydrazine-rich mixture is further processed as a useful byproduct or recycled as process fuel (e.g. combusted to heat distillation column 14).

The distillate mixture D1 of hydrazine hydrate, hydroxyethylhydrazine, and water from the distillation column 14 is then fed into a second reactor 16, where it is mixed with a nitrate feedstock F4. The nitrate feedstock F4 includes nitric acid or ammonium nitrate (AN). Although either may be used, ammonium nitrate facilitates a reduction of side reactions in the reactor 14. The hydrazine hydrate and hydroxyethylhydrazine react with the nitrate to produce a product mixture F5 that includes hydrazine hydrate, hydrazinium nitrate (HN), hydroxyethylhydrazine (HEH), and hydroxyethylhydrazinium nitrate (HEHN). The constituent fractions of the product mixture F5 can be adjusted by controlling the ratio between the distillate mixture D1 and the nitrate feedstock F4. For example, the ratio includes an equilibrium mixture (i.e., a mixture where the chemical species inter-react until an equilibrium distribution is established, whereafter the constituent relative fractions remain constant) of hydrazine hydrate, HN, HEH, HEHN, ammonia, ammonium nitrate, and water according to the Equation II below.

$$Equation II$$

$${N}_{2} {H}_{4} \cdot {H}_{2} O + HEH + {H}_{2} O + AN \to {N}_{2} {H}_{4} \cdot {H}_{2} O + HN + HEH + HEHN + {NH}_{3} + AN + {H}_{2} O$$

To further illustrate, the reaction of 4.4 parts by mole of a distillate D1 comprising 54.6% N₂H₄·H₂O, 22.7% HEH, 22.7% and H₂O by mole with one part of feedstock F4 comprising 100% ammonium nitrate will yield an equilibrium product mixture F5 approximately comprising 44.0% N₂H₄·H₂O, 0.8% HN, 0.1% HEH, 16.4% HEHN, 17.2% NH₃, 4.2% AN, and 17.3% H₂O by mole. This reaction may preferably be carried out at or near ambient temperature and pressure, as it is neither pressure nor temperature sensitive.

The product mixture F5 is then processed through one or more distillation columns 18. The distillate D2 that exits at the top of the distillation column 18 essentially includes hydrazine hydrate, water, and ammonia (if ammonium nitrate is used as the nitrate source). As the distillate D2 constituents are removed, the equilibrium of the remaining mixture in the distillation column 18 shifts to produce the desired final mixture M of hydrazine, hydroxyethylhydrazinium nitrate, and hydrazinium nitrate, which exits the bottom of the distillation column 18 according to Equation III below. (Due to its high basicity, a negligible portion of the hydroxyethylhydrazine remains, as essentially all of it is protonated to become hydroxyethylhydrazinium nitrate.) For example, accounting for 10% loss of ethylene oxide as bis(hydroxyethyl) hydrazine and 15% loss of hydrazinium nitrate during distillations, a final mixture M having a molar composition of 65% hydrazine, 27% HEHN, and 8% HN can be produced from 1.15 moles hydrazine hydrate, 0.297 moles of ethylene oxide, and 0.350 moles of ammonium nitrate per mole of the final mixture M. In further examples, the final mixture M has a molar composition of 50-75% hydrazine, 15-35% hydroxyethylhydrazinium nitrate, and 5-15% hydrazinium nitrate.

$$Equation III$$

$${N}_{2} {H}_{4} \cdot {H}_{2} O + HN + HEH + HEHN + {NH}_{3} + AN + {H}_{2} O \to {[ {NH}_{3} + {H}_{2} O ]}_{distillate} + {[ {N}_{2} {H}_{4} + HEHN + HN ]}_{b o t t o m s}$$

Ammonia recovered from the distillate D2 can be harvested as a useful byproduct or recycled as a process fuel, and hydrazine hydrate recovered from the distillate D2 can be recycled and used in the feedstock F1.

In further example, rather than conducting the reaction in reactor 12 and the distillation in distillation column 14 in-line with the reaction in reactor 16 and distillation in distillation column 18 to provide the mixture of hydrazine hydrate, hydroxyethylhydrazine, and water of distillate D1 that is used as an input into the reactor 16, a pre-mixed feedstock of hydrazinium nitrate, hydroxyethylhydrazine, and optionally water is used as the input into the reactor 16. This in essence severs the manufacturing process to include only the steps associated with the reactor 16 and the distillation column 18.

It is to be appreciated that additional process steps and intermediate distillations may be used in-between any of the steps disclosed herein to separate out constituents that are not intended to be present in the final mixture M. The method disclosed herein may be implemented as a batch process or as a continuous-flow process, and over a broad range of scales from laboratory use to industrial production. It is also to be appreciated that process parameters, such as the excess hydrazine hydrate-to-ethylene oxide ratio, the types of distillation column types, reflux ratios, operating temperatures, operating pressures, or other parameters may be varied in order to tailor process cost and/or efficiency, for example in accordance with variations in feedstocks or energy costs.

Although a combination of features is shown in the illustrated examples, not all of them need to be combined to realize the benefits of various embodiments of this disclosure. In other words, a system designed according to an embodiment of this disclosure will not necessarily include all of the features shown in any one of the FIGURES or all of the portions schematically shown in the FIGURES. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

What is claimed is:

1. A method comprising:
reacting a mixture of hydrazine hydrate and hydroxyethylhydrazine with a nitrate to produce a product mixture that includes hydrazine hydrate, hydrazinium nitrate, hydroxyethylhydrazine, and hydroxyethylhydrazinium nitrate; and
distilling the product mixture to produce a final mixture that includes hydrazine, hydroxyethylhydrazinium nitrate, and hydrazinium nitrate.

2. The method as recited in claim 1, wherein the nitrate is selected from the group consisting of ammonium nitrate and nitric acid.

3. The method as recited in claim 1, wherein the nitrate includes ammonium nitrate.

4. The method as recited in claim 1, wherein the nitrate includes nitric acid.

5. The method as recited in claim 1, wherein the nitrate is ammonium nitrate.

6. The method as recited in claim 1, wherein the nitrate is nitric acid.

7. The method as recited in claim 1, wherein the mixture of hydrazine hydrate and hydroxyethylhydrazine is produced by first reacting ethylene oxide and an excess of hydrazine hydrate to yield an intermediate mixture that includes the hydrazine hydrate, the hydroxyethylhydrazine, and bis(hydroxyethyl) hydrazine.

8. The method as recited in claim 7, further including distilling the intermediate mixture to substantially remove the bis(hydroxyethyl) hydrazine from the intermediate mixture to produce the mixture of hydrazine hydrate and hydroxyethylhydrazine.

9. The method as recited in claim 1, wherein the final mixture has a molar composition of 50-75% hydrazine, 15-35% hydroxyethylhydrazinium nitrate, and 5-15% hydrazinium nitrate.

10. A method comprising:
producing a mixture of hydrazine hydrate and hydroxyethylhydrazine by reacting ethylene oxide and an excess of hydrazine hydrate to yield an intermediate mixture that includes the hydrazine hydrate, the hydroxyethylhydrazine, and bis(hydroxyethyl) hydrazine;
distilling the intermediate mixture to substantially remove the bis(hydroxyethyl) hydrazine from the intermediate mixture to produce the mixture of hydrazine hydrate and hydroxyethylhydrazine;
reacting the mixture of hydrazine hydrate and hydroxyethylhydrazine with a nitrate to produce a product mixture that includes hydrazine hydrate, hydrazinium nitrate, hydroxyethylhydrazine, and hydroxyethylhydrazinium nitrate; and
distilling the product mixture to yield a final mixture that includes hydrazine, hydroxyethylhydrazinium nitrate, and hydrazinium nitrate.

11. The method as recited in claim 10, wherein the nitrate is selected from the group consisting of ammonium nitrate and nitric acid.

12. The method as recited in claim 10, wherein the nitrate includes ammonium nitrate.

13. The method as recited in claim 10, wherein the nitrate includes nitric acid.

14. The method as recited in claim 10, wherein the nitrate is ammonium nitrate.

15. The method as recited in claim 10, wherein the nitrate is nitric acid.

16. The method as recited in claim 10, wherein a molar ratio of the the excess of hydrazine hydrate to the ethylene oxide is from 2:1 to 9:1.
